(19) United States
(12) Patent Application Publication
(10) Pub. No.: US 2022/0367024 A1
(43) Pub. Date: Nov. 17, 2022

(54) ALGORITHM FOR IMPROVING OVARIAN STIMULATION

(71) Applicant: Dmitri Dozortsev, Houston, TX (US)

(72) Inventor: Dmitri Dozortsev, Houston, TX (US)

(21) Appl. No.: 17/874,932

(22) Filed: Jul. 27, 2022

Related U.S. Application Data
(63) Continuation-in-part of application No. 17/736,773, filed on May 4, 2022.
(60) Provisional application No. 63/186,135, filed on May 9, 2021.

(51) Int. Cl. G16H 20/10; G01N 33/76

(52) U.S. Cl. CPC G16H 20/10; G01N 33/76

(57) ABSTRACT

An approach is disclosed for achieving improved egg quality and simplified management in patient undergoing controlled ovarian stimulation. Information, including endogenous FSH level, is received about the patient. An optimal duration for the controlled ovarian stimulation is estimated. A target level of FSH is estimated to achieve the optimal duration. Pharmacological formulations are administered to change FSH to achieve the target level of FSH. Resulting FSH is compared to target FSH level. The amount of pharmacological formulation is adjusted when the target FSH level is not achieved; and oocyte's final maturation is triggered after achieving the optimal duration.

If an Application Data Sheet (ADS) has been filed for this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

For purposes of the USPTO extra-statutory requirements, the present application constitutes a utility application related to and claims the benefit of priority from U.S. Provisional Patent Application No. 63186,135 filed on May 9, 2021.

### BACKGROUND

The present invention relates to the field of obstetrics and gynecology, specifically to achieving a control over ovulation for infertility treatment.

### SUMMARY

According to one embodiment of the invention, there is provided a method for achieving improved egg quality and simplified management in patient undergoing controlled ovarian stimulation. Information, including endogenous of follicle stimulating hormone (FSH) level, is received about the patient. An optimal duration is estimated for the controlled ovarian stimulation. A target level of FSH in the patient is estimated to achieve the optimal duration. A pharmacological formulation changing FSH level in circulation to achieve the target level of FSH is administered. Resulting FSH in the patient is measured. The resulting FSH is compared to the target FSH level. The amount of the pharmacological formulation is adjusted when the resulting FSH level does not match the target FSH level. When the optimal duration is achieved, triggering oocyte's final maturation is triggered.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present invention will be apparent in the non-limiting detailed description set forth below.

### BRIEF DESCRIPTION OF THE DRAWINGS

The present invention may be better understood, and its numerous objects, features, and advantages made apparent to those skilled in the art by referencing the accompanying drawings, wherein:

FIG. 1 depicts ovarian ovulation cycle;

FIG. 2 depict legacy Al algorithm for ovarian stimulation;

FIG. 3 depicts changing control by egg over follicle over time;

FIG. 4 depicts epidemiological evidence for impact of duration of ovarian cycle on egg quality;

FIG. 5 depicts epidemiological evidence that variations of duration of ovulatory cycle reflect variations in duration of follicular phase;

FIG. 6 depicts retrospective analysis of clinical data demonstrating improvement for chance of pregnancy with increased duration of ovarian stimulation;

FIG. 7 depicts ultrasonographic evidence of improving chance of pregnancy by increasing duration of follicular phase;

FIG. 8 depicts embodiment 1, a process for improving oocytes quality by increasing a follicular phase of an ovarian cycle of a patient;

FIG. 9 depicts embodiment 2, a process for improving oocytes quality by increasing a duration of follicular phase of an ovarian cycle of a patient;

FIG. 10 depicts embodiment 3, a process for achieving optimal duration of controlled ovarian stimulation in patient undergoing assisted reproduction;

FIG. 11 depicts embodiment 4, a process for achieving optimal duration of controlled ovarian stimulation in patient undergoing assisted reproduction;

FIG. 12 depicts embodiment 5, a process for achieving optimal duration of controlled ovarian stimulation in patient undergoing assisted reproduction;

FIG. 13 depicts embodiment 6, a process for tuning optimal duration of controlled ovarian stimulation in patient undergoing assisted reproduction; and

FIG. 14 depicts a schematic view of a processing system wherein the methods of this invention may be implemented.

### DETAILED DESCRIPTION

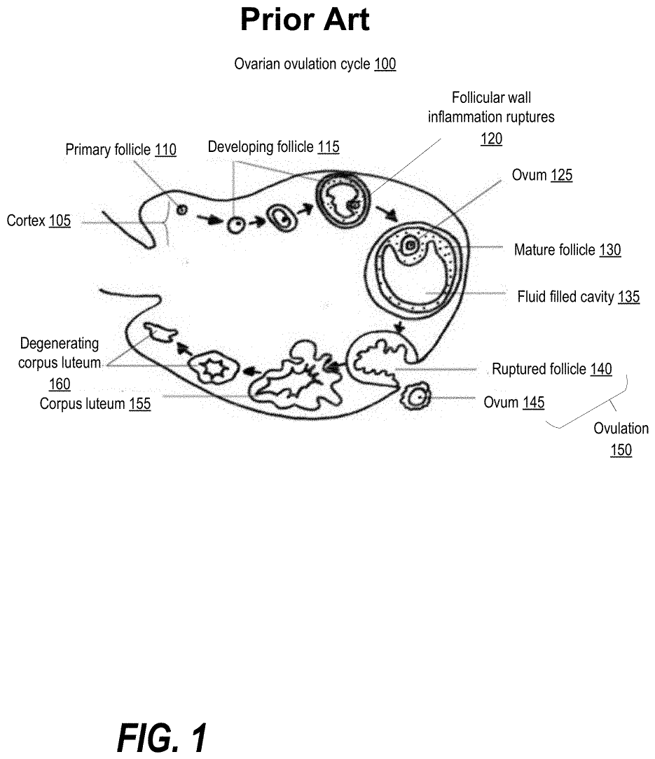

It is generally accepted that egg quality (aka developmental competency or maturity) is the most important predictor for the success of human reproduction, including assisted human reproduction. Egg matures within ovarian follicle. The final stage of egg maturation takes place during so-called follicular phase, which last about 2 weeks. Toward the end of follicular phase, follicle ruptures releasing the egg and making it available for fertilization (FIG. 1). Since the egg is very small and is contained within the follicle, it is impossible to directly measure its quality. However, it is possible to measure the size of the follicle that contains the egg using ultrasound and also by measuring level of estradiol, produced by follicle, in blood. Since the size and the amount of estradiol produced by follicle around time of ovulation during natural follicular phase is known, those measurements may be used as benchmarks to infer the state of developmental competency of the eggs during controlled ovarian stimulation. This approach works reasonably well for the majority of cases and has been clinically useful for several decades since clinical IVF inception to yield so-called chromosomally mature eggs. Chromosomal maturity of an egg can be easily discerned by presence of the so-called first polar body. Presence of first polar body is generally accepted sign that ovarian stimulation was successful since chromosomally mature egg was obtained. However, it has been known for a long time, that despite some correlation between, on one hand, follicle size and estradiol level and on the other hand, quality of the egg found in that follicle, the correlation is not strong, despite presence of polar body (1). Furthermore, it has also been well known that within the group of eggs with the first polar body eggs present there is vast variation in their ability to develop once fertilized.

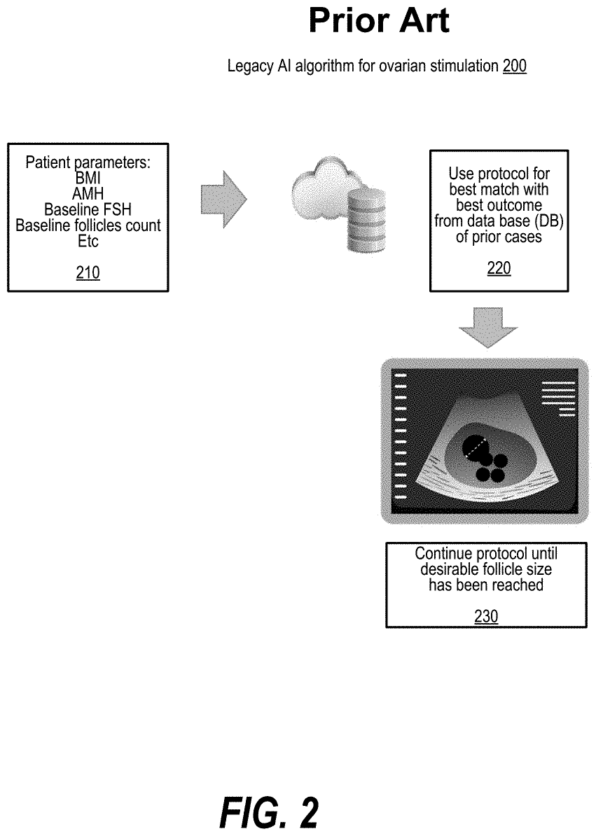

Still, for the lack of better markers, state-of-the-art artificial intelligence (AI) algorithms for ovarian stimulation use the same approach (FIG. 2) tracking the size of the follicle and estradiol to predict the best day for harvesting egg. Therefore, they suffer the same limitations as state-of-the-art “manual” practice.

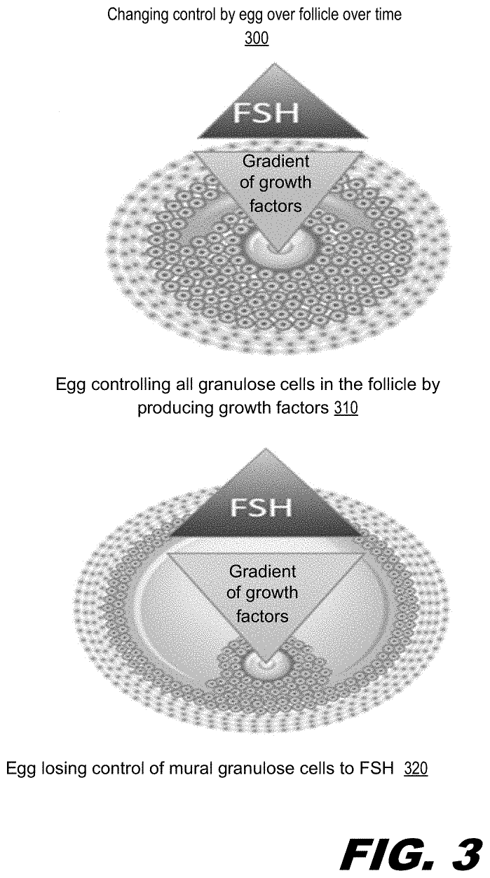

The likely reason for lack of correlation between parameters of follicle (size and estradiol activity) and egg quality is that by the time when follicle is recruited into follicular phase, egg no longer controls the follicle (FIG. 3). From that perspective, measuring the follicle to predict egg quality is not unlike attempting to predict term delivery measuring pregnant woman's belly: there will be a correlation, but it will be weak, since developing fetus does not control the amount of amniotic fluid.

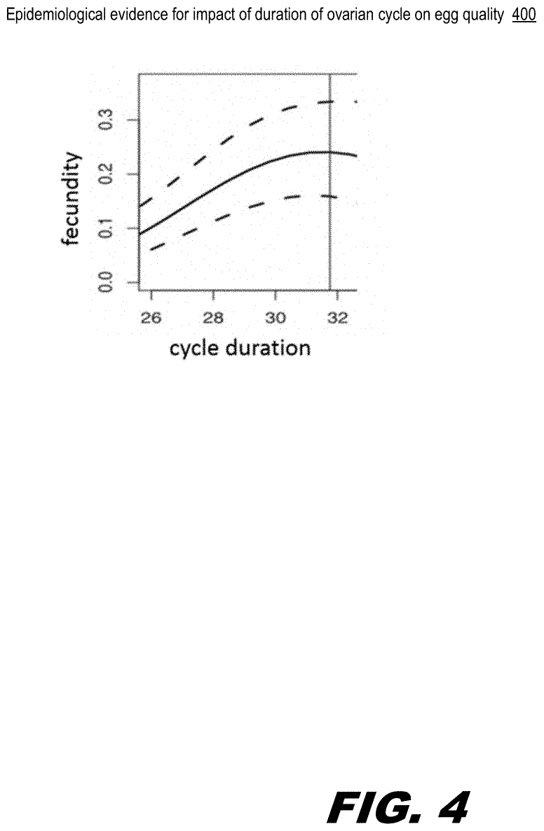

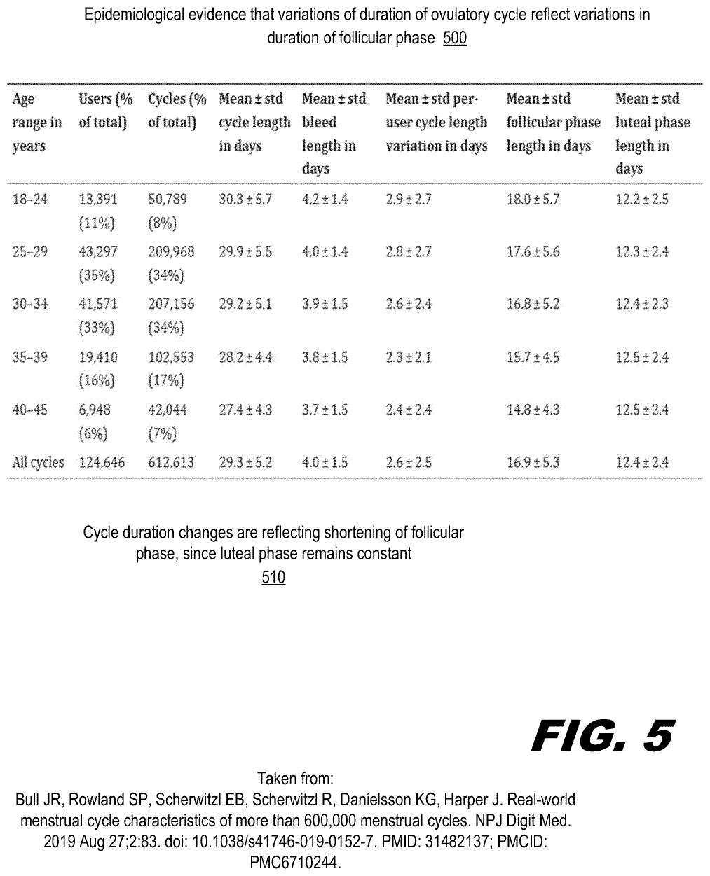

At the same time, it has been well known that the duration of a natural ovarian cycle has strong predictive power for probability of pregnancy (FIG. 4). Reliable epidemiological data suggest that reduction of the cycle by mere 4 days reduces fecundity by staggering 50%. Importantly, since duration of luteal phase (part of the cycle after ovulation) remains relatively constant (FIG. 5), overall duration of the ovarian cycle is determined by follicular phase (2). Thus, shortening of follicular phase by 4 days has catastrophic impact on chance of conception. Until recently, it was believed that short follicular phase is predictive of lower fecundity because follicle ovulates prematurely at a smaller size when it does not have enough cells to produce sufficient amount of estradiol and progesterone that are required to prime uterus for implantation.

However, there is an alternative possibility, that the observed drop in fecundity with shortening follicular phase is due to insufficient time for an egg to acquire developmental competence. This is like a prematurely born child as opposed to a child born at term. Accordingly, “term follicular phase” designates such duration of follicular phase (natural or induced) that is expected to produce a competent egg.

In order to test the possibility of existence of term follicular phase, a database of prior cases of ovarian stimulation was analyzed to determine whether longer stimulation produces better quality eggs. These types of studies were carried out before by other investigators, who failed to find a correlation, but all of them had a fatal bias: in order to prove or disprove the influence of the duration of the follicular phase on the outcome, one must only include those cases when the duration is a completely independent variable. In other words, the duration of stimulation must not be a derivative of patient's response to ovarian stimulation. Prior studies did not fulfill that requirement by including cases when follicular phase is extended because patient failed to respond appropriately (3).

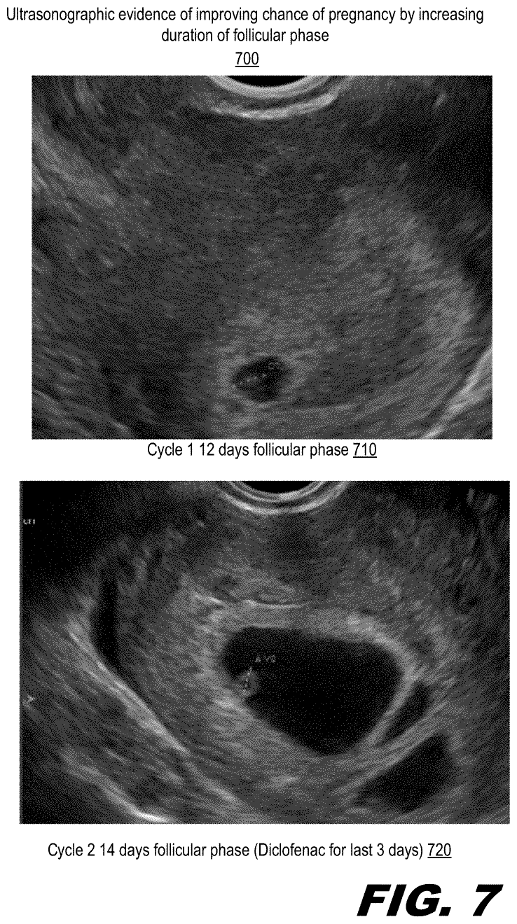

When only cases where ovarian stimulation was extended due to random reasons, for example, where patient eggs could not be retrieved on time because the clinic was closed or due to clerical error, a statistically significant improvement in probability of pregnancy was found (FIG. 7).

Next, five patients were chosen in different clinics where prior stimulation, targeting follicle size, was not successful and every known approach had been tried to achieve pregnancy. The target duration of extended follicular phase was chosen based on the epidemiologically known duration of follicular phase in the female during the peak of her reproductive potential—16.5 days. It is not always realistic to achieve 16.5 days due for a variety of reasons. It should also be noted that in some cases follicle phase duration longer than 16.5 days is required to achieve better outcome.

Several approaches were used to increase the duration of follicular phase. In some cases, the amount of injected of follicle stimulating hormone (FSH) was reduced, to slow down follicle expansion or estradiol was administered to lower endogenous FSH. In others, non-steroid anti-inflammatory drug, diclofenac was applied, that extends the follicular phase, providing the egg with more time to gain the developmental competence. Yet, in other cases, the patient was maintained in the artificial follicular phase despite rupture and loss of the largest follicle so that the remaining follicles had additional time to nurture the egg, until the target duration of the follicular phase was achieved. Ovarian stimulation that targets physiological duration of follicular phase in fertile female is referred to as Term Stimulation™ (TS).

In all five cases, four of which were considered desperate, ongoing pregnancy or/and live births were achieved at the time of this disclosure. Three representative cases follow.

In the very first case, a female who had five prior IVF attempts with no embryo development after fertilization, became pregnant, her follicular phase was extended and delivered normal child term.

This case is of particular interest because it demonstrates that not only TS achieves better results than conventional stimulation, but also that it seems to be improving oocyte quality compared to the natural cycle.

Case report 1. (with Albert Ichmelyan, MD, PhD). Patient 35 years old. Gravida (G) −0, Para (P) −0

Cycle 1. Long protocol, follicles-4, Metaphase (M) II-5, 2 pronuclei (PN)-1, arrest on day 3 with a high rate of fragmentation

Cycle 2. Natural. MII-1, 2PN-0

Cycle 3. Natural. MII-1, 2PN-0

Cycle 4. Natural. MII-1, 2PN-0

Cycle 5. Natural. MII-1, 2PN-1, arrested at 2 cells stage

Cycle 6. TS, follicles-7, MII-5, MI (matured in vitro to MII)-2, IVF-MII-2 with donor sperm 2PN-0. Intracytoplasmic sperm injection (ICSI)-MII-5 with husband sperm—2PN-4. Blastocysts—2 (from oocytes injected with husband's sperm). The patient delivered a healthy boy, 9.5 lb, 22.4″ at 40 weeks.

Case report 2. (with Dr. Tralik, MD, PhD and Michael Allon, MD). Patient 34 years, G-0, P-0.

Clomid, Timed intercourse. The patient's medication was identical in Cycle 1 and 2

Cycle 1. The follicular phase was 12 days. Three follicles developed. A single fetal sack was seen on ultrasound at 10 weeks, small for gestational age. The pregnancy ended in miscarriage at 12 weeks.

Cycle 2. TS: the duration of the follicular phase was extended under the protection of diclofenac (for the last 3 days) to 14 days. Three follicles were recruited, and three fetal sacks were seen on the ultrasound at 10 weeks. Two had the adequate size for gestational age. Pregnancy spontaneously reduced to twins, which were delivered at 37 weeks by c-section.

Case report 3. Patient 31 years old. G-0, P-0. IVF. Two identical ovarian stimulation regimens two months apart.

Cycle1. Stimulation—9 days (effective follicular phase—13 days). From 13 fertilized oocytes developed only 3 blastocysts.

Cycle 2. TS: exactly the same stimulation protocol as in her first cycle with the only difference that it was started 5 days later so that the effective follicular phase became 18 days as opposed to 13 in the first cycle. In the second retrieval, the patient had 19 fertilized oocytes, 13 developed into excellent quality blastocysts and all of them, except 1 were chromosomally normal.

A new theory should explain at least one paradox, which does not have a satisfactory explanation under the current paradigm. One of such paradoxes is the unexplainably low oocyte quality in very young IVF patients and another one similar problem in patients with polycystic ovaries syndrome (PCOS). The two phenomena have been puzzling physicians for many years.

The concept of term maturation provides a simple and very plausible explanation for both paradoxes. Young patients and those with PCOS have unusually large number of follicles at the start of the follicular phase and respond to hormonal stimulation with most of them recruited into the cycle. Because of an unusually large number of growing follicles, estradiol is rising at a higher pace and also the follicles reach ovulatory size earlier than in the natural cycle. This makes it necessary to stop ovarian stimulation and harvest oocytes earlier than from other patients. This shortening of follicular phase, according to term maturation theory, would be expected to negatively impact eggs quality, creating a false impression that the eggs were intrinsically poor quality. In truth, the eggs were probably perfectly good oocytes, which simply did not get enough time to acquire full development potential.

The duration of the follicular phase is mainly determined by two conditions: 1. the pace of the follicle's expansion in size and 2. The ability of ovarian cortex to accommodate the follicle's expansion without follicle inflaming and rupturing. The elasticity and other properties of the ovarian cortex, which determine follicle's maximum size before rupture cannot be controlled. Therefore, the only remaining tools that can be used to control the duration of the follicular phase is the pace of the follicle's expansion and inflammation: the slower it expands and the lower the inflammation, the longer it will take to reach the critical size where follicle begins to disintegrate. The inflammation control can be achieved by non-steroid anti-inflammatory drugs (NSAD), such as diclofenac.

The pace of follicle's expansion is controlled by amount of follicle stimulating hormone (FSH) in circulation. During ovarian stimulation it is comprised of woman's own FSH and amount of injected FSH as determined by physician. Amount of endogenous FSH in circulation can also be controlled by administering estradiol, which lowers the amount of FSH produced by patient. Algorithms are disclosed to achieve TS that relies on the amount of FSH in circulation. The goal of TS is to achieve duration of ovarian stimulation, which is effect is artificial follicular phase, that would match physiological duration of follicular phase in her prime of fertility, while recruiting the maximum number of follicles.

Since due to its relatively long half-life, injected FSH compounded over time, it may achieve such high level that will adversely affects oocytes quality (possibly by reallocating cumulus cells from nursing egg to producing estradiol). This may require continued adjustment of the level of FSH in circulation to keep it within a target range.

Even though FSH is a primary driver of follicle expansion, during controlled ovarian stimulation other medications are also necessary to prevent premature ovulation, premature follicle rupture, and mitigate exceedingly high level of estradiol, which may be harmful to a female.

According to one embodiment of the invention, there is provided a method for improving egg quality and simplifying management of controlled ovarian stimulation in patient undergoing assisted reproduction. The method relies on achieving the duration of ovarian stimulation matching the duration of follicular phase of a female during the peak of fertility. The algorithm predicts target level of FSH in the patient to achieve the desired (optimal) duration. A pharmacological formulation is administered to bring FSH level in circulation to the target level. Resulting FSH is measured in the patient to determine whether the target FSH level is achieved. An amount of said pharmacological formulation is adjusted when the target FSH level is not achieved or when it does not support optimal duration. Responsive to achieving the desired duration, oocyte's final maturation is triggered.

FIG. 1 illustrates an ovarian cycle 100. The ovarian cortex 105 functions as a site of storage and growth of the follicles that contain eggs at different stages their developmental competency. The primary follicle 110 forms in the cortex 105 or antrum and develops by absorbing nutrients in the cortex 105. Stages of the developing follicle 155 are depicted where the follicle grows, the wall of the follicle becomes inflamed to the point where the follicular wall inflammation ruptures 120 expelling the ovum 125 leaving the mature follicle 130 which was expelled from the fluid filled cavity 135. The ruptured follicle 140 with the expelled ovum 145 is called ovulation 150. The ruptured follicle 140 becomes the corpus luteum 155 that produces progesterone required for fertilized egg implantation and If implantation fails, corpus luteum degrades to become the degenerating corpus luteum 160.

FIG. 2 depicts legacies AI algorithm for ovarian stimulation 200. At step 210, patient parameters: body mass index (BMI), anti-mullerian hormone (AMH), baseline FSH, baseline follicles count, etc. At step 220, the process uses protocol for best match with best outcome from data base (DB) of prior cases. At step 230, the process continues protocol until desirable follicle size has been reached. A typical fully mature follicle size is about 20 mm.

FIG. 3 depicts changing control by egg over follicle over time 300. 310 depicts egg controlling all granulose cells by producing growth factors in the follicle while the follicle remains small (pre-antral) and prevents the induction of FSH receptors in granulosa. The inverted triangular represents the gradient of oocyte produced growth factors includes growth differentiation factor (GDF), bone morphogenetic protein (BMP), Small Mothers Against Decapentaplegic (SMAD)

320 depicts an antral follicle at the recruitment into ovulatory cycle, when an egg is losing control over the follicle to FSH, because the gradient of growth factors becomes diluted and mural granulose cells develop FSH receptors. From this time onwards, egg's maturation is at the mercy of the follicle (or an REI). The inverted triangular represents the gradient of oocyte produced growth factors includes: GDF-9, BMP-15, SMAD.

FIG. 4. 400 depicts epidemiological evidence for impact of duration of ovarian cycle on egg quality. The fecundity drops catastrophically, when the duration of the ovarian cycle is shorten by 4 days.

FIG. 5. 500 depicts epidemiological evidence that variations of duration of ovulatory cycle reflect variations in duration of follicular phase. In 510, cycle duration changes reflect shortening of follicular phase, since luteal phase remains constant. This suggest that the catastrophic drop in FIG. 4, must be due to shortening follicular phase.

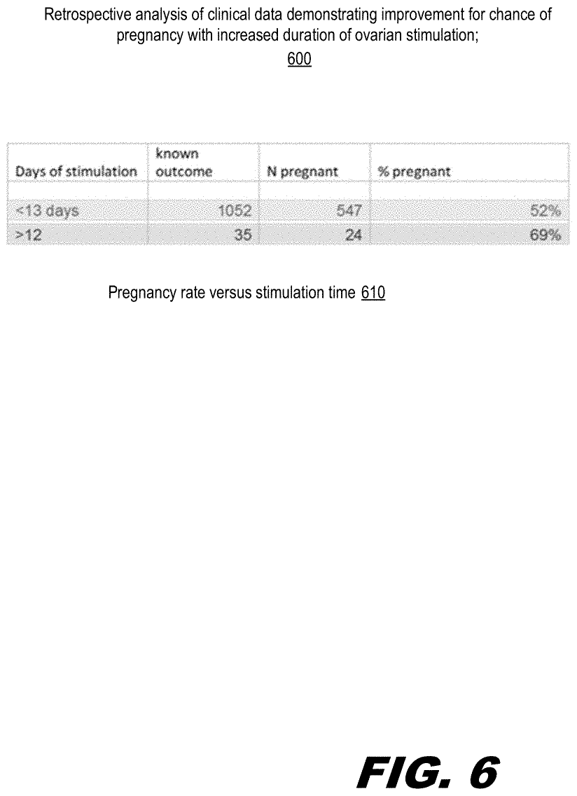

FIG. 6. 600 depicts retrospective analysis of clinical data demonstrating improvement for chance of pregnancy with increased duration of ovarian stimulation. In 610, pregnancy rates in IVF cases drop with shortening of ovarian stimulation. This suggests that drop of fecundity in FIG. 4 and FIG. 5 are due to changes in egg quality rather than due to smaller corpus luteum.

FIG. 7. 700 depicts ultrasound pictures effects from diclofenac in a case study with two cycles of treatment with following parameters: 34 years, clomid, timed intercourse 700. 710 depicts cycle 1 with 12 days in follicular phase. Implantation sack is small for gestational age and resulted in spontaneous miscarriage. 720 depicts cycle 2 with 14 days follicular phase (Diclofenac for last 3 days). Patient has three implantation sacks, of which two resulted in delivery of two healthy children.

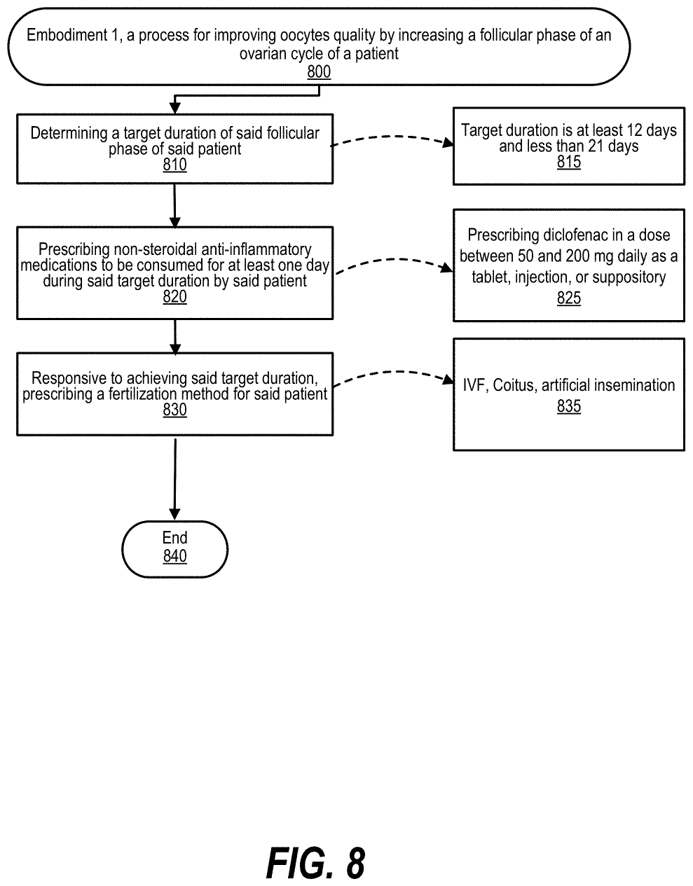

FIG. 8 processing commences at 800 and shows the steps taken by embodiment 1, a process for improving oocytes quality by increasing a follicular phase of an ovarian cycle of a patient. At step 810, the process determines a target duration of said follicular phase of said patient. At step 815, the process target duration is at least 12 days and less than 21 days. At step 820, the process prescribes non-steroidal anti-inflammatory medications to be consumed for at least one day during said target duration by said patient. At step 825, the non-steroidal anti-inflammatory medication is diclofenac in a dose between 50 and 200 mg daily as a tablet, injection, or suppository is prescribed. The method may restrict consumption of said diclofenac by said patient to a last quarter of said follicular phase. The said ovarian cycle may be a natural cycle or a controlled ovarian stimulation cycle. At step 830, the process continues responsive to achieving said target duration, by prescribing a fertilization process for said patient. At step 835, the fertilization process is, for example, IVF, coitus, or artificial insemination. FIG. 8 processing thereafter ends at 840.

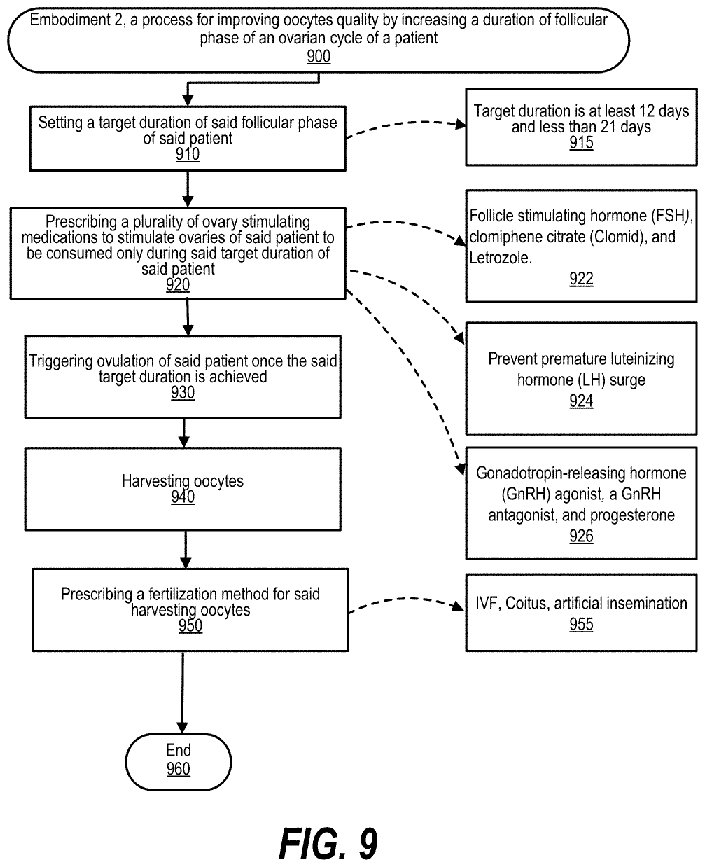

FIG. 9 processing commences at 900 and shows the steps taken by embodiment 2, a process for improving oocytes quality by increasing a duration of follicular phase of an ovarian cycle of a patient. At step 910, the process sets a target duration of said follicular phase of said patient. At step 915, the target duration is at least 12 days and less than 21 days. At step 920, the process prescribes a plurality of ovary stimulating medications to stimulate ovaries of said patient to be consumed only during said target duration of said patient. At step 922, examples of said plurality of ovary stimulating medications include follicles stimulating hormone (FSH), clomiphene citrate (Clomid), and Letrozole. At step 924, examples of said plurality of ovary stimulating medications include preventing premature luteinizing hormone (LH) surge. At step 926, examples of said plurality of ovary stimulating medications include gonadotropin-releasing hormone (GnRH) agonist, a GnRH antagonist, and progesterone. At step 940 oocytes are harvested. At step 950, the process prescribes a fertilization process for said harvesting oocytes. At step 955, examples of said fertilization process include IVF, coitus, and artificial insemination. FIG. 9 processing thereafter ends at 960.

These methods to improve egg quality by extending follicular phase to the duration equal to that of duration of follicular phase in the females at the peak of reproductive potential is called Term Maturation in line with term pregnancy.

Ovarian stimulation that targets achieving Term Maturation is called Term Stimulation™. Once the desirable size of the follicles is reached, an ovulation trigger drug, usually Human chorionic gonadotropin (hCG) or Lupron, is administered to induce final stages of oocytes maturation and ovulation. After that, an intercourse or assisted reproductive procedure is carried out.

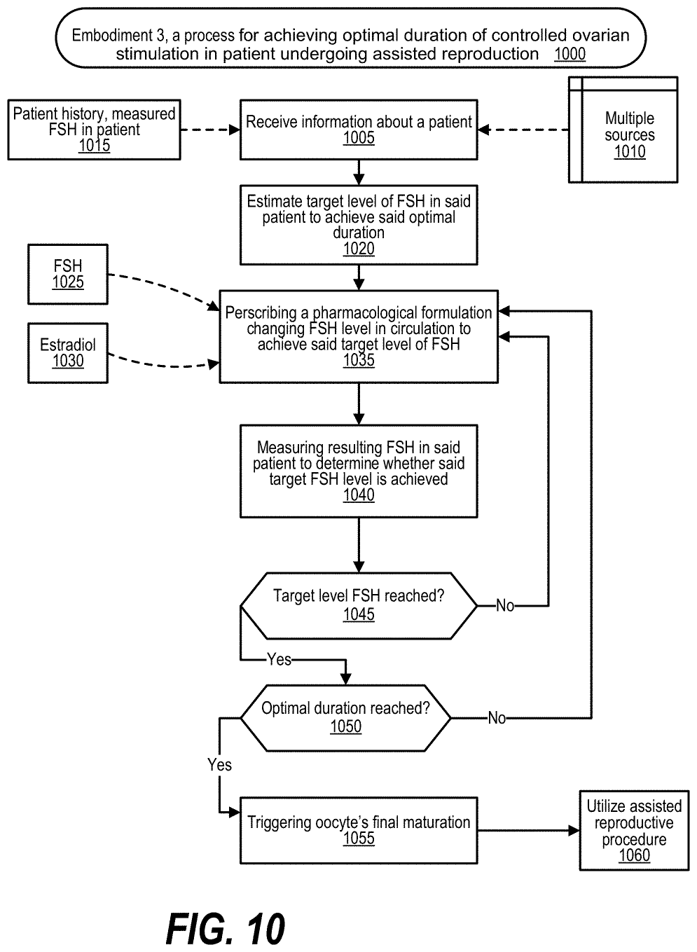

FIG. 10 processing commences at 1000 and shows the steps taken by embodiment 3, a process for achieving optimal duration of controlled ovarian stimulation in patient undergoing assisted reproduction. At step 1005, the process receives information about a patient. The received information may be from multiple sources 1010. The said received information may be, for example, but not limited to importing medical records, data collected directly from the patent, such as, by drawing blood for AMH, estradiol, and other fertility markers, measuring weight and height, receiving answers to questions from the patient, and the like. The received information may include measured FSH in patient 1015. 1020, the process estimates target level of FSH in said patient to achieve said optimal duration. The estimation may be based on case histories retrieved from a database of case histories based on a base match of demography of the patient to cases in the database of case histories. At step 1035, a pharmacologic formulation is administered to the patient to change FSH level in circulation to achieve said target level of FSH. The administered pharmacologic formation may be, for example, FSH 1025 to increase the FSH level in circulation. Alternatively, the estradiol 1030 may be administered to lower the FSH level in circulation. At step 1040, the process measures resulting FSH in said patient to determine whether said target FSH level is achieved. The process determines as to whether target level FSH reached (decision 1045). If target level FSH is not reached, then decision 1045 branches to the ‘No’ branch which loops back to step 1035. This looping continues until the target level FSH is reached, at which point decision 1045 branches to the ‘Yes’ branch exiting the loop. The process determines as to whether optimal duration reached (decision 1050). If optimal duration is not reached, then decision 1050 branches to the ‘No’ branch which loops back to 1035. This looping continues until the optimal duration is reached, at which point decision 1050 branches to the ‘Yes’ branch exiting the loop. At step 1055, the process triggers oocyte's final maturation. At step 1060, the process utilizes an assisted reproductive procedure. The utilization may include, for example, freezing eggs or fertilization. Examples of said fertilization process include IVF, coitus, and artificial insemination.

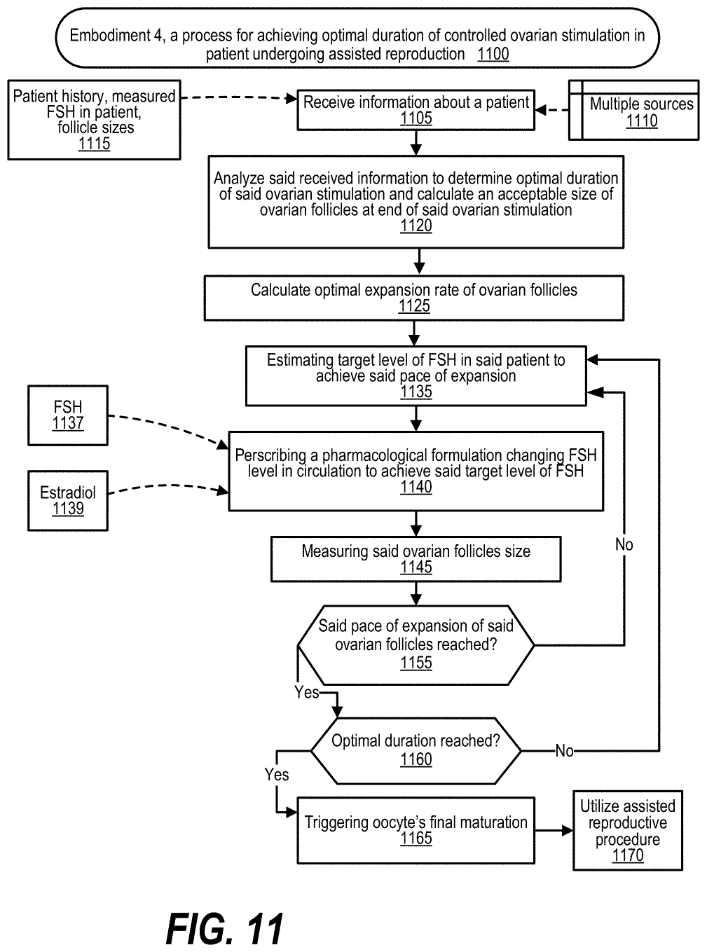

FIG. 11 processing commences at 1100 and shows the steps taken by embodiment 4, a process for achieving optimal duration of controlled ovarian stimulation in patient undergoing assisted reproduction. At step 1105, the process receives information about a patient. The said received information may be from multiple sources 1110. The sources may include, for example, but not limited to importing medical records, data collected directly from the patent, such as, by drawing blood, measuring weight and height, receiving answers to questions from the patient, and the like. The received information may include demography, measured FSH in patient, and follicle sizes 1115. At step 1120, the process analyzes said received information to determine optimal duration of said ovarian stimulation and calculate an acceptable size of ovarian follicles at end of said ovarian stimulation. At step 1125, the process calculates optimal expansion rate of ovarian follicles. At step 1135, a target level of FSH in said patient to achieve said pace of expansion is estimated. At step 1140, a pharmacologic formulation is administered to the patient to change FSH level in circulation to achieve said target level of FSH. The administered pharmacologic formation may be, for example, FSH 1137 to increase the FSH level in circulation. Alternatively, estradiol 1139 may be administered to lower the FSH level in circulation. At step 1145, the process measures said ovarian follicles size. In an embodiment, the target maximum size of the follicles minus the current size is the total growth size. The total growth size should be spread out through the optimal duration period of time. So, the total growth size divided by the optimal duration is the desired expansion rate. These calculations may be adjusted as updated measurements are received and the treatment time progresses. The process determines as to whether said pace of expansion of said ovarian follicles reached (decision 1155). If said pace of expansion of said ovarian follicles is not reached, then decision 1155 branches to the ‘No’ branch which loops back to 1135. This looping continues until the said pace of expansion of said ovarian follicles is reached, at which point decision 1155 branches to the ‘Yes’ branch exiting the loop. The process determines as to whether optimal duration reached (decision 1160). If optimal duration is not reached, then decision 1160 branches to the ‘No’ branch which loops back to 1135. This looping continues until the optimal duration is reached, at which point decision 1160 branches to the ‘Yes’ branch exiting the loop. At step 1165, the process triggers oocyte's final maturation. At 1170, the process utilizes an assisted reproductive procedure. The utilization may include, for example, freezing eggs or fertilization. Examples of said fertilization process include IVF, coitus, and artificial insemination.

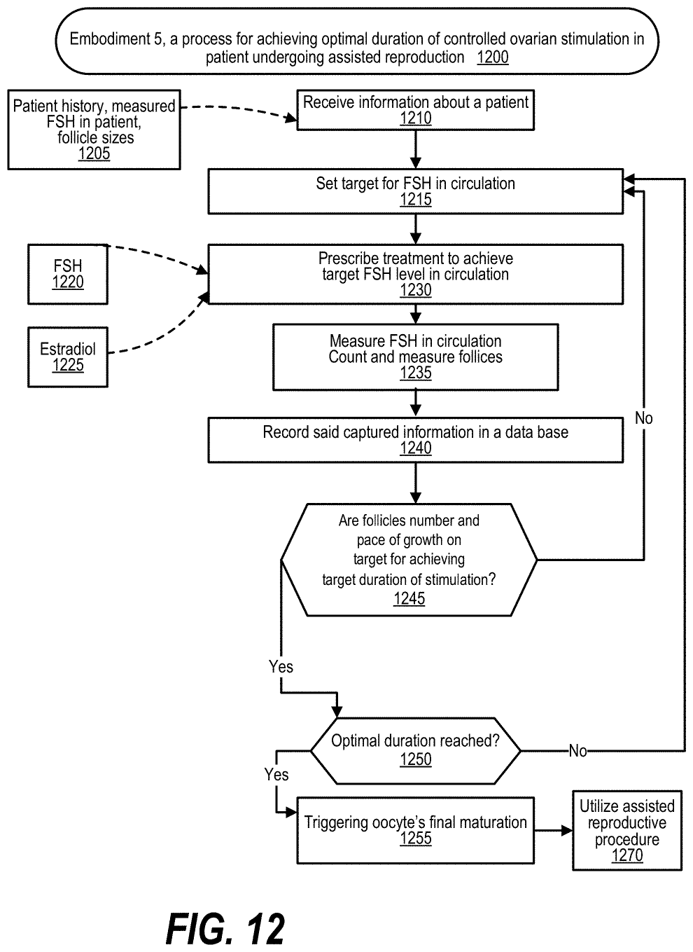

FIG. 12 processing commences at 1200 and shows the steps taken by embodiment 5, a process for achieving optimal duration of controlled ovarian stimulation in patient undergoing assisted reproduction. At step 1210, the process receives information about a patient. The said received information may include demography, measured FSH in patient, and follicle sizes 1205. The sources may include, for example, but not limited to importing medical records, data collected directly from the patent, such as, by drawing blood, measuring weight and height, receiving answers to questions from the patient, and the like. At step 1215, a target for FSH in circulation is set. At step 1230, a pharmacologic formulation is administered to the patient to change FSH level in circulation to achieve said target level of FSH in circulation. The administered pharmacologic formation may be, for example, FSH 1220 to increase the FSH level in circulation. Alternatively, the estradiol 1225 may be administered to lower the FSH level in circulation. At step 1240, the process measures resulting FSH in said patient to determine whether said target FSH level is achieved. At step 1235, the process measures FSH in circulation and counts and measure follicles. At step 1240, the process records said captured information in a data base (DB). The process determines as to whether are follicles number and pace of growth on target for achieving target duration of stimulation (decision 1245). If follicles number and pace of growth are not on target for achieving target duration of stimulation, then decision 1245 branches to the ‘No’ branch which loops back to 1215. This looping continues until follicles number and pace of growth are on target for achieving target duration of stimulation, at which point decision 1245 branches to the ‘Yes’ branch exiting the loop. The process determines as to whether optimal duration reached (decision 1250). If optimal duration is not reached, then decision 1250 branches to the ‘No’ branch which loops back to 1215. This looping continues until the optimal duration is reached, at which point decision 1250 branches to the ‘Yes’ branch exiting the loop. At step 1255, the process triggers oocyte's final maturation. At step 1260, the process utilizes an assisted reproductive procedure. The utilization may include, for example, freezing eggs or fertilization. Examples of said fertilization process include IVF, coitus, and artificial insemination.

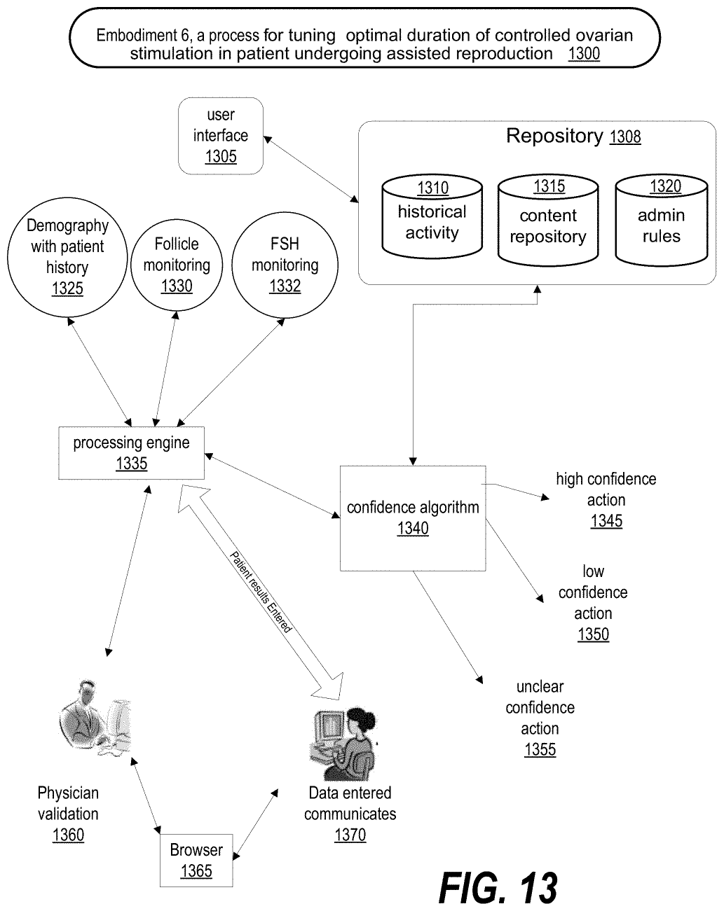

FIG. 13 processing commences at 1300 and shows the steps taken by a embodiment 6, a process for tuning optimal duration of controlled ovarian stimulation in patient undergoing assisted reproduction. Information from a plurality of patients is processed by, for example, embodiment 5 as depicted in FIG. 12. The information, such as, demography with patient history 1325, follicle monitoring 1330, and FSH monitoring 1332 is input into processing engine 1335. The processing engine 1335 may have feedback and input from others. In an example embodiment, a browser 1365 may be used a physician 1360 to communicate with the processing engine 1335. Similarly, a browser 1365 may be used to communicate patent results identifying relevant data entered and communicated 1370 with the processing engine 1335. The processing engine 1335 may be part of an artificial intelligence model that uses confidence algorithm 1340, deep analysis, or a deep neural network to classify and enter data into repository 1308.

The data, may be, for example, derived from interactions via instant messages between the physician 1360 and patient. The patient results may be entered and communicated to the processing engine 1335. The data entered and communicated 1370 may be by lab technician or received from medical records indicating lab results. The processing engine 1335 utilizes confidence algorithm 1340 which interfaces with repository 1308. The repository 1308 may have various elements. The elements may include, but are not limited to, for example, historical activity 1310 that captures other patent histories recorded in the past for similar content stored in content repository 1315, and admin rules 1320 that are followed when interfacing with repository 1308. The repository 1308 may have its own user interface 1305 or an application programming interface (API) allowing modification to the support to the admin rules 1320. The confidence algorithm 1340 may apply various admin rules 1320 based on different optimization rules. The rules could be by patent demography, for example, by age group, medical history, or any other patient specific factors, and the like. Using the admin rules 1320, the confidence algorithm 1340 may utilize some type of statistical assessment to predict if a change to the categorization of a case history should be made. When the confidence algorithm 1340 determines that categorization has a high probability of improving categorization, the confidence algorithm 1340 performs a high confidence action 1345, such as, for example, but not limited to, adding an additional demography class to content repository 1315 under a predicted improvement However, if the confidence algorithm 1340 determines that adding the additional demography class to the content repository 1315 has a low probability of improving automated FSH adjustment, the confidence algorithm 1340 performs a low confidence action 1350, such as, for example, but not limited to, eliminating the proposed additional demography class. If the confidence algorithm 1340 determines that adding a demography class has an unclear probability of improving automated FSH adjustment, the confidence algorithm 1330 performs an unclear confidence action 1355, such as, for example, but not limited to, recording related information in historical activity 1310. The confidence algorithm 1340 may have an Artificial Intelligence (AI) component that learns which terms are relevant and utilizes a feedback loop adding new evaluations and new results to determine which terms referring to demography are relevant. The feedback loop would have expected advantages, such as, speeding up processing time, improving matching case studies. The communication between physician 1360 or the data enterer 1370 and the patient could be, for example and without limitation, one or more of the following: verbal, text, text selections, short message service (SMS), instant message, interactive voice response, numeric keyboard, browser 1365 GUI elements, forums, social media, texting, smart phone application, and the like. The patient contact may even be a voice response system service that itself uses artificial intelligence (AI) to communicate with patient.

It must also be appreciated that optimal duration of stimulation for a specific patient can be estimated using epidemiological data, database of prior cases or parameters of the patient's history.

Furthermore, although follicle's size measurement is incorporated into some of the embodiments, it is expected that once sufficient number of cases has been processed, follicle's measurement will no longer be required. This will greatly simplify ovarian stimulation, since ultrasound visits to the clinic will become unnecessary. In some embodiments, the entire monitoring of ovarian stimulation will be performed at the comfort of patient's home.

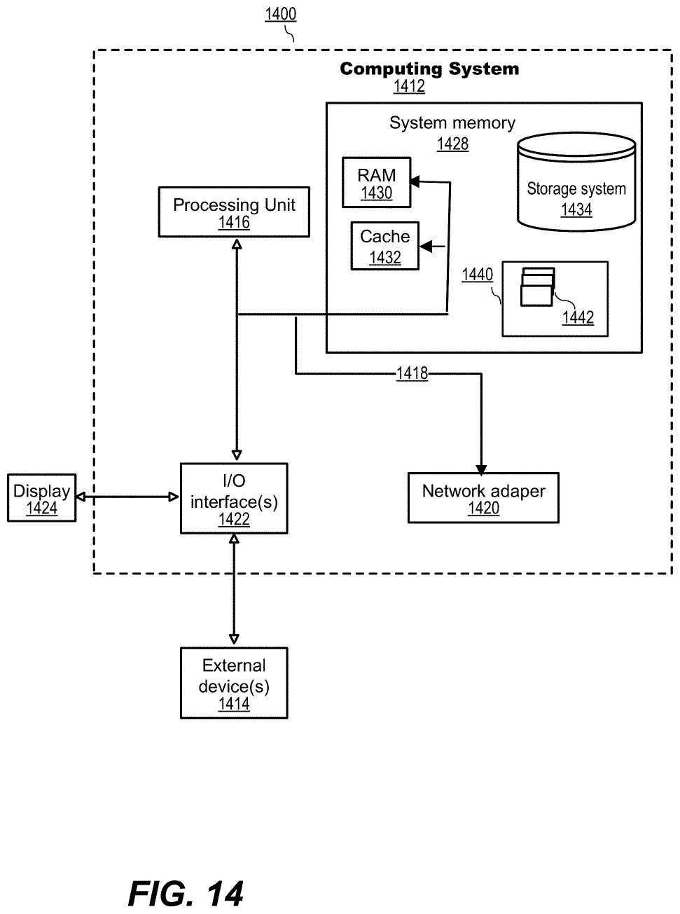

Referring to FIG. 14, a schematic view of a processing system 1400 is shown wherein the methods of this invention may be implemented. The processing system 1400 is only one example of a suitable system and is not intended to suggest any limitation as to the scope of use or functionality of embodiments of the invention described herein. Regardless, the system 1400 can implement and/or performing any of the functionality set forth herein. In the system 1400 there is a computer system 1412, which is operational with numerous other general purpose or special purpose computing system environments or configurations. Examples of well-known computing systems, environments, and/or configurations that may be suitable for use with the computer system 1412 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system 1412 may be described in the general context of computer system-executable instructions, such as program modules, being executed by a computer system. Generally, program modules may include routines, programs, objects, components, logic, data structures, and so on that perform tasks or implement abstract data types. The computer system 1412 may be practiced in distributed cloud computing environments where tasks are performed by remote processing devices that are linked through a communications network. In a distributed cloud computing environment, program modules may be in both local and remote computer system storage media including memory storage devices.

As shown in FIG. 14, the computer system 1412 in the system environment 1400 is shown in the form of a general-purpose computing device. The components of the computer system 1412 may include, but are not limited to, a set of one or more processors or processing units 1415, a system memory 1428, and a bus 1418 that couples various system components including the system memory 1428 to the processor 1415.

The bus 1418 represents one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA) bus, the Micro Channel Architecture (MCA) bus, the Enhanced ISA (EISA) bus, the Video Electronics Standards Association (VESA) local bus, and the Peripheral Component Interconnects (PCI) bus.

The computer system 1412 typically includes a variety of computer system readable media. Such media may be any available media that is accessible by the computer system 1412, and it includes both volatile and non-volatile media, removable and non-removable media.

The system memory 1428 can include computer system readable media in the form of volatile memory, such as random-access memory (RAM) 1430 and/or a cache memory 1432. The computer system 1412 may further include other removable/non-removable, volatile/non-volatile computer system storage media. By way of example only, a storage system 1434 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to the bus 1418 by one or more data media interfaces. As will be further depicted and described below, the system memory 1428 may include at least one program product having a set (e.g., at least one) of program modules 1442 that are configured to carry out the functions of embodiments of the invention.

A program/utility 1440, having the set (at least one) of program modules 1442, may be stored in the system memory 1428 by way of example, and not limitation, as well as an operating system, one or more application programs, other program modules, and program data. Each of the operating systems may have one or more application programs, other program modules, and program data or some combination thereof, and may include an implementation of a networking environment. The program modules 1442 generally carry out the functions and/or methodologies of embodiments of the invention as described herein.

The computer system 1412 may also communicate with a set of one or more external devices 1414 such as a keyboard, a pointing device, a display 1424, a tablet, a digital pen, etc. wherein these one or more devices enable a user to interact with the computer system 1412; and/or any devices (e.g., network card, modem, etc.) that enable the computer system 1412 to communicate with one or more other computing devices. Such communication can occur via Input/Output (I/O) interfaces 1422. These include wireless devices and other devices that may be connected to the computer system 1412, such as, a USB port, which may be used by a tablet device (not shown). Still yet, the computer system 1412 can communicate with one or more networks such as a local area network (LAN), a general wide area network (WAN), and/or a public network (e.g., the Internet) via a network adapter 1420. As depicted, a network adapter 1420 communicates with the other components of the computer system 1412 via the bus 1418. It should be understood that although not shown, other hardware and/or software components could be used in conjunction with the computer system 1412. Examples include, but are not limited to microcode, device drivers, redundant processing units, external disk drive arrays, RAID systems, tape drives, and data archival storage systems, etc.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While particular embodiments have been shown and described, it will be obvious to those skilled in the art that, based upon the teachings herein, that changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

### REFERENCES

1. Shapiro B S, Rasouli M A, Verma K, Raman A, Garner F C, Aguirre M, Kaye L, Bedient C. The effect of ovarian follicle size on oocyte and embryology outcomes. Fertil Steril. 2022 June; 117(6):1170-1176. doi: 10.1016/j.fertnstert.2022.02.017. Epub 2022 Mar. 30. PMID: 35367061.
2. Bull J R, Rowland S P, Scherwitzl E B, Scherwitzl R, Danielsson K G, Harper J. Real-world menstrual cycle characteristics of more than 600,000 menstrual cycles. NPJ Digit Med. 2019 Aug. 27; 2:83. doi: 10.1038/s41746-019-0152-7. PMID: 31482137; PMCID: PMC6710244.
3. Irani M, Canon C, Robles A, Maddy B, Gunnala V, Qin X, Zhang C, Xu K, Rosenwaks Z. No effect of ovarian stimulation and oocyte yield on euploidy and live birth rates: an analysis of 12 298 trophectoderm biopsies. Hum Reprod. 2020 May 1; 35(5):1082-1089. doi: 10.1093/humrep/deaa028. PMID: 32348476.

What is claimed is:

1. A method for achieving improved egg quality and simplified management in patient undergoing controlled ovarian stimulation comprising:
receiving information, including endogenous FSH level, about said patient;
estimating optimal duration for said controlled ovarian stimulation;
estimating target level of FSH in said patient to achieve said optimal duration;
prescribing a pharmacological formulation changing FSH level in circulation to achieve said target level of FSH;
measuring resulting FSH in said patient;
comparing said resulting FSH to said target level of FSH;
adjusting amount of said pharmacological formulation when said resulting FSH does not match target level of FSH; and
responsive to achieving said optimal duration, triggering oocyte's final maturation.

2. A method of claim 1, further comprising:
analyzing said received information to determine acceptable size of ovarian follicles at end of said ovarian stimulation;
calculating target pace of said ovarian follicle expansion to not exceed said acceptable size of said ovarian follicles at said end of said optimal duration;
estimating target level of FSH in said patient to achieve said pace of ovarian follicles expansion;
measuring said ovarian follicles after a period of time;
comparing said measured pace of follicles expansion to said target pace of expansion;
adjusting the amount of said pharmacological formulation and said target FSH level if the said pace of follicles expansion does not match said target pace of expansion;
measuring estradiol level in blood;
responsive to detecting estradiol level in blood exceeding safe level, prescribing estradiol lowering medication; and
responsive to identifying risk of premature follicle rupture, prescribing anti-inflammatory formulations to delay follicles rupture.

3. The method of claim 1, wherein said optimal duration is about equal to duration of physiological follicular phase.

4. The method of claim 1, wherein said target level of FSH provides said pace of ovarian follicles expansion to grow to a size of about 20 mm by said end of said optimal duration.

5. The method of claim 1, wherein said target level of FSH recruits maximum number of follicles.

6. The method of claim 1, wherein said cumulative level of FSH is below 25 IU/L.

7. The method of claim 1, wherein said acceptable size of the follicles is a range.

8. The method of claims 1, wherein said target level of FSH is a range.

9. The method of claim 1, wherein said optimal duration is a range.

10. The method of claims 1, wherein said pharmacological formulation contains FSH.

11. The method of claims 1, wherein said pharmacological formulation contains hCG.

12. The method of claims 1, wherein said pharmacological formulation contains an agent lowering FSH.

13. The method of claim 12, wherein said agent lowering FSH is estradiol.

14. The method of claim 1, further comprising:
prescribing a fertilization procedure.

15. The method of claim 14, wherein the fertilization procedure is selected from a group consisting of coitus, intrauterine insemination, and in vitro fertilization.

16. The method of claim 1, further comprising:
capturing said oocytes.

17. The method of claim 1, further comprising:
performing steps of said measuring and said adjusting iteratively until said optimal duration is reached.

18. The method of claim 1, wherein said analyzing patient information further comprises:
finding a plurality of best matches in a database of cases;
extracting statistical data from said plurality of best matches; and
applying said extracted statistical data as input to said analyzing.

19. The method of claim 18, further comprising:
capturing case studies for said achieving optimal duration of controlled ovarian stimulation for a plurality of patients P (P1, P2, . . . , Pn) undergoing assisted reproduction; and
storing said captured case studies in said database of cases.

20. The method of claim 19, further comprising:
training an artificial intelligence model utilizing said case studies to map said plurality of patients P (P1, P2, . . . , Pn) with corresponding demographics D (D1, D2, . . . , Dn) to a corresponding treatment plans TP (TP1, TP2, . . . , TPn) to achieve said target level of FSH;
receiving a new patient Pk with a demography Dk;
applying the trained artificial intelligence model to said new patient Dk having said demography Dk to identify said demography treatment plan TPk for said new patient Pk; and
prescribing said identified said demography treatment plan TPk to said new patient Pk.
